(19) United States
(12) Patent Application Publication
(10) Pub. No.: US 2017/0209640 A1
(43) Pub. Date: Jul. 27, 2017

(54) SHOWER WASHING SYSTEM AND METHOD

(71) Applicant: Elizabeth Felder, Houston, TX (US)

(72) Inventor: Elizabeth Felder, Houston, TX (US)

(21) Appl. No.: 15/004,900

(22) Filed: Jan. 23, 2016

(51) Int. Cl. A61M 3/02

(52) U.S. Cl. CPC A61M 3/0233; B05B 1/18

(57) ABSTRACT

A system and method for cleansing a cavity in a living body is disclosed. The system uses pressurized water from a plumbing system and directs a portion of that water into a douche pipe for cleaning the cavity. The system includes a shower attachment member that is configured to collect water from the plumbing. Also included is a douche pipe configured to direct the flow of water toward the opening of the cavity in a rearward axial direction so as to let the pressure of the water pull bacteria or infections directly to the opening for removal. The douche pipe includes at least one port that is oriented in a non-perpendicular orientation.

### BACKGROUND

1. Field of the Invention

The present application relates to a system and method of medical treatment, and more particularly to a system and method for a douche used to wash portions of the body while in the shower.

2. Description of Related Art

A douche is a device used to introduce a stream of water into the body for medical or hygienic reasons. Douche usually refers to vaginal irrigation, the rinsing of the vagina, but it can also refer to the rinsing of any body cavity, such as the anal and nasal cavity. Traditionally a fluid bag is used to hold a volume of water. The bag is elevated relative to the cavity to be cleansed. The fluid in the bag is in communication, via a tube, with a douche pipe. The tube drains the fluid into the pipe that is placed within the cavity. The water is expelled from the pipe through a series of ports in the pipe so as to induce a washing effect.

The vaginal cavity holds a balance of good and bad bacteria. At times the ratio of bad bacteria can increase leading to infections or other problems with the vagina. The cause of the imbalance can be from any number of factors. It is believed that the cleansing of the vaginal cavity can assist in restoring bacterial balance and the removal of infections.

One disadvantage of present kits is the need for a separate bag to be filled to hold the water or fluid used. The bag is limited in the amount of fluid it can hold and there are times where an adequate hanging location is unavailable. An additional disadvantage of present douche kits involves the pipe. The ports on the pipe are typically aligned so as to be perpendicular to the surface of the pipe. With this design, the water is configured to discharge perpendicular to the axis of the pipe. The impact of the water on the cavity walls disperses water in all directions. The water momentum is lost after impact. There is limited, if any, force in the water to withdraw the infection. Steps are taken by the individual to maneuver the pipe in and out of the cavity to help pull out the infection. Also, the individual may rotate, tilt, and twist the pipe as needed. A new design for the pipe is necessary that allows for the direct use of the water pressure to aid in pulling out the infections and other bacteria from the cavity.

Although some strides have been with respect to douche systems, considerable shortcomings remain.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a side view of a shower washing system according to the preferred embodiment of the present application.

FIG. 2 is a side view of a douche pipe in the shower washing system of FIG. 1.

FIG. 3 is a side view of an exemplary douche pipe used in the shower washing system of FIG. 1 and depicting the direction of flow of water exiting the system.

FIG. 4 is a side view of an alternative embodiment of the douche pipe of FIG. 2.

FIG. 5 is a side view of the shower washing system of FIG. 1 modified for use with a fluid bag.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system and method in accordance with the present application overcomes one or more of the above-discussed problems commonly associated with conventional douche systems. The system is configured to replace the use of a fluid bag with a shower attachment member configured to seal around a shower head so as to provide a constant and unlimited amount of fluid for douching. Additionally, the system is configured to use an improved douche pipe that directs the flow of the fluid in a more axial direction toward the exterior of the cavity. The direction of flow is toward the base of the douche pipe and the tubing and is meant to provide improved performance by pushing the infection out of the cavity. The water flow directs the bacteria toward the opening of the cavity for discharging. Furthermore, an additional advantage of the present system is the ability to filter the liquid used and to regulate the flow rate of the liquid. These and other unique features of the device are discussed below and illustrated in the accompanying drawings.

The system and method will be understood, both as to its structure and operation, from the accompanying drawings, taken in conjunction with the accompanying description. Several embodiments of the system may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system and method of the present application is illustrated in the associated drawings. The assembly includes a shower attachment member, a tube, and a douche pipe. The shower attachment member is configured to surround a portion of a shower head and capture water from the shower head and pass it through the tube. The tube passes the water down to the douche pipe for use within a designated cavity. The douche pipe is configured to expel the water under pressure in a rearward axial direction so as to actively withdraw any infection or bacteria out of the opening of the cavity. A filter and a regulating valve may also be used to provide additional safety and control when used.

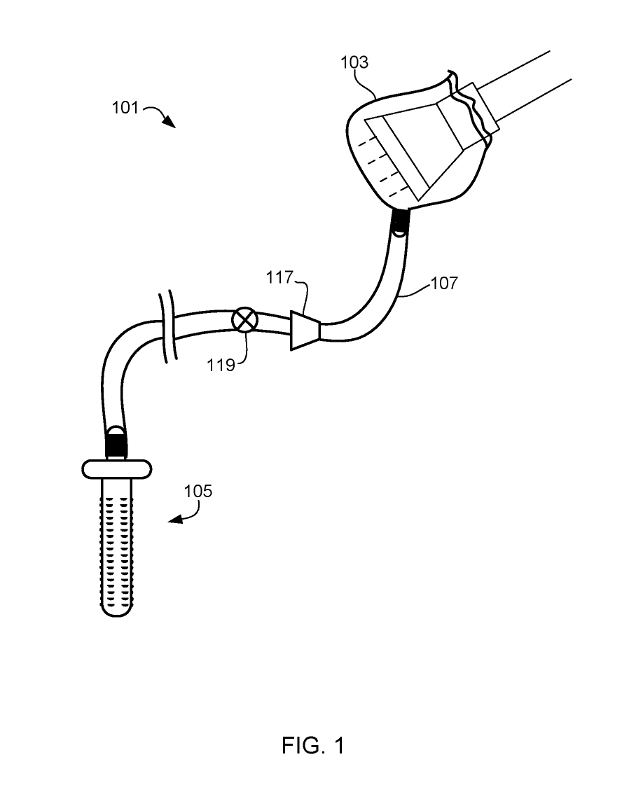

Referring now to the drawings wherein like reference characters identify corresponding or similar elements in form and function throughout the several views. FIG. 1 illustrates the general configuration of shower washing system 101. System 101 includes a shower attachment member 103, a douche pipe 105, and a tube 107.

System 101 is configured to cleanse a cavity in a living body by using a supply of liquid predominantly provided by water directed to a shower head. Use of water from a shower head allows for an unlimited and endless amount water such that the need to swap fluid bags is moot. This leads to greater convenience to the user. Additionally, system 101 is configured to direct the water exiting the douche pipe into a rearward direction toward the tubing. This is advantageous because it eliminates the need of the user to have to manipulate the orientation of the douche pipe within the cavity in order to allow for the water to push/pull the bacteria/infections toward the opening of the cavity.

System 101 may be used in any cavity of a living body. Primarily this is thought of as a human body. System 101 may be used with animals as well if necessary. Various cavities may be cleansed with system 101 and/or douche pipe 105, namely at least a vaginal cavity, nasal cavity, anal cavity, and so forth. Each of the elements used in system 101 are selectively detachable for ease of cleaning and also interchangability.

System 101 is configured to use water provided from indoor plumbing lines. Typically this if done through a shower or even possibly a bath tub. The water is pressurized and provides a steady supply of water. Shower attachment member 103 is configured to selectively engage the stream of water in the plumbing at any location. Example locations may be through the line itself via a nipple or release valve where tubing 107 may be selectively attached. Another example allows for member 103 to collect water as it exits the shower head itself. As seen in FIG. 1, member 103 is configured to releasably couple to the shower head and surround at least a portion of it. In this manner, member 103 is configured to collect a portion of the water exiting the shower head for use within system 101.

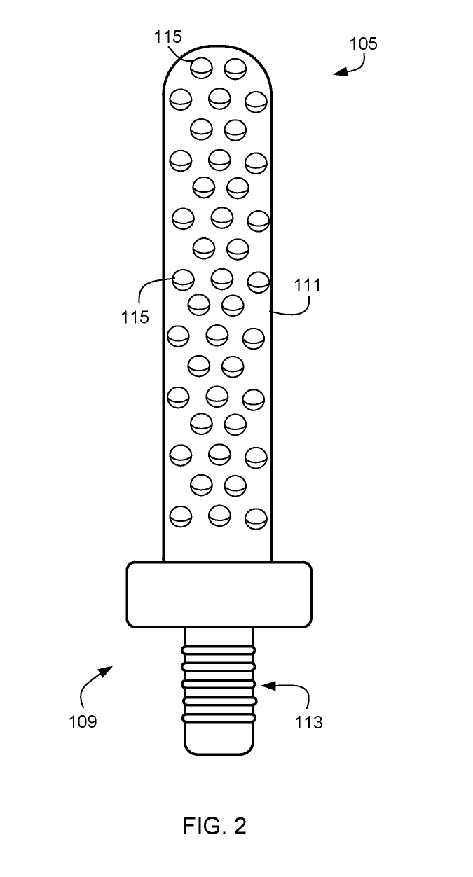

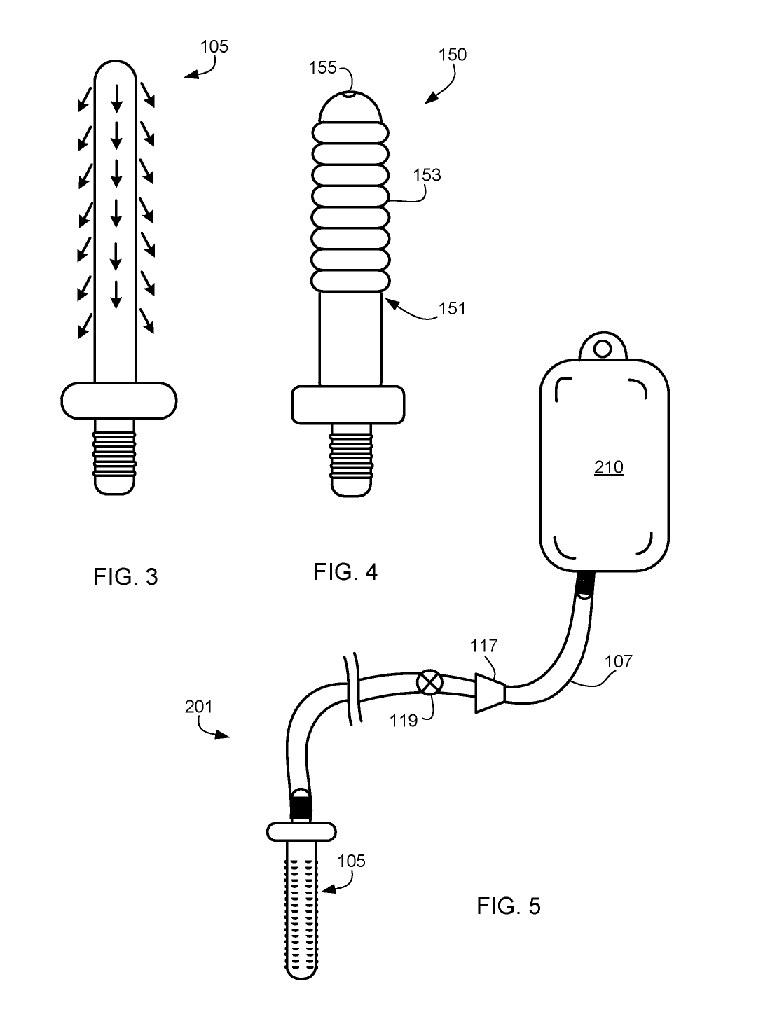

Tubing 107 is configured to carry the water from member 103 to that of douche pipe 105. Tubing 107 is in fluid communication with both. Douche pipe 105 is seen more clearly in FIGS. 2 and 3 in the drawings. Pipe 105 is configured to selectively introduce liquid (i.e. water) into a cavity of the body. Pipe 105 is configured to include a base portion 109 and a shaft 111. Base portion 109 includes a fitting 113 for attachment of tube 107. Fitting 113 has an internal passage that allows for the passing of fluid from tube 107 into shaft 111. Fitting 113 is also configured to optionally include ribbing to increase the grip with tube 107. Shaft 111 is configured to internally receive fluid passing through fitting 113 and thereby eject this fluid through at least one of a plurality of ports 115. Ports 115 are dispersed along a portion of shaft 111. Ideally ports 115 are dispersed around the circumference of shaft 111 to permit an even distribution of fluid exiting. Ports 115 are apertures that pass through the wall of shaft 111 to create a channel from an internal passage in communication with fitting 113 to the exterior of the shaft.

As seen in FIG. 3 in particular, pipe 105 is designed such that at least one of the plurality of ports 115 are aligned so as to direct the flow of fluid exiting shaft 111 in a rearward direction toward base portion 109. At least one of the plurality of ports 115 are formed in shaft 111 at a non-perpendicular angle relative to the axis of shaft 111. By aligning at least one of the plurality of ports 115 rearward, the pressurized fluid exiting shaft 111 passes predominantly along the walls of the cavity in the direction of the cavity opening, thereby resulting in the water pulling/pushing any bacteria/infection outside of the cavity opening. Because of this alignment, the user is also not required to have to manipulate the orientation of the douche pipe in the cavity in order to attempt to perform the same result. The arrows in FIG. 3 represent an exemplary direction of fluid flow exiting at least one of the plurality of ports 115.

It is understood that pipe 105 is configured to allow for the ergonomical fitting within the cavity. Shaft 111 may be formed in various contours to allow for better fit and control by the user. The strait design is not herein limiting to pipe 105 but is shown as merely an example of one type of contour.

Referring back to FIG. 1, system 101 may optionally include a filter 117 and a valve 119. Filter 117 is in communication with the fluid in tube 107. Filter 117 is configured to cleanse the liquid prior to entering pipe 105. Filter 117 may be selectively removed and interchanged as necessary. Interchanging of filter 117 allows a user to more actively regulate the level of filtration within system 101. It is understood that one or more filters may be used. Additionally, filter 117 is not herein limited to a location in direct communication with tube 107. Other locations are permitted, such as within member 103 and pipe 105.

Valve 119 is in communication with the fluid passing through tube 107 as well. Similarly to that of filter 117, valve 119 may be located in communication with member 103 and pipe 105 as opposed to tube 107 as shown. Valve 119 is configured to regulate the flow of fluid within system 101. Valve 119 is configured to turn on/off the flow of liquid through system 101. Additionally, valve 119 is configured to allow the user to selectively choose the amount of flow passing through system 101 such that system 101 may be able to accommodate different flow rates from the plumbing and shower heads.

Referring now also to FIG. 4 in the drawings, an alternative embodiment of pipe 105 is shown. Pipe 150 is interchangeable with that of pipe 105 within system 101. Pipe 150 is configured to include a shaft 151 having one or more ribs 153. Ribs 153 may be oriented around the exterior of shaft 151 in any direction and have any size. In FIG. 4, ribs 153 wrap around the circumference of shaft 151 perpendicular to the axis of shaft 151. The ribs are useful in gripping the cavity of the body. Ideally this type of pipe would be useful in the anal cavity of a human. A port 155 is located at the tip of shaft 151 for the exiting of the fluid.

Referring now also to FIG. 5 in the drawings, system 101 is illustrated in a modified condition wherein member 103 is replaced by a fluid bag 210. System 201 is similar in form and function to that of system 101 except in that member 103 is replaced with fluid bag 210. The shower washing system of the present application is adaptable to work or operate with legacy liquid bag douche systems. Any portion of system 101 may be used with legacy douche systems (i.e. pipes 105/150, tubing 107, filter 117, valve 119)

The current application has many advantages over the prior art including at least the following: (1) means to provide for a constant supply of liquid to douche so as to eliminate the need to refill a bag; (2) pre-pressurized water; (3) improved douche pipe that directs the flow of the fluid in a more axial direction toward the exterior opening of the cavity; and (4) ability to filter and regulate the flow rate of the fluid passing within the system.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A shower washing system, comprising:
a shower attachment member configured to capture a portion of the water being supplied to a shower head;
a douche pipe configured to selectively introduce liquid partially including water from the shower head into a cavity of the body; and
a tube in communication with the shower attachment member and the douche pipe, the tube configured to maintain fluid communication;
wherein the douche pipe is configured to direct the flow of liquid toward the opening of the cavity in a rearward axial direction.

2. The system of claim 1, wherein the shower attachment member is releasably coupled to a shower head.

3. The system of claim 1, wherein the shower attachment member wraps around the shower head.

4. The system of claim 1, wherein the shower attachment member captures all the water exiting the shower head.

5. The system of claim 1, wherein the fluid exiting the douche pipe is under pressure such that the pressure is used to direct the expulsion of bacteria out the opening of the cavity without movement of the douche pipe.

6. The system of claim 1, further comprising:
a filter in communication with the fluid and the tube, the filter configured to cleanse the fluid prior to reaching the douche pipe.

7. The system of claim 1, further comprising:
a valve in communication with the tube and configured to regulate the flow of fluid into the douche pipe.

8. The system of claim 1, wherein the tube is selectively detachable from the shower attachment member and the douche pipe.

9. A shower washing system, comprising:
a shower attachment member configured to capture a portion of the water being supplied to a shower head;
a douche pipe configured to selectively introduce liquid partially including water from the shower head into a cavity of the body;
a tube in communication with the shower attachment member and the douche pipe, the tube configured to maintain fluid communication;
a filter in communication with the fluid and the tube, the filter configured to cleanse the fluid prior to reaching the douche pipe; and
a valve in communication with the tube and configured to regulate the flow of fluid into the douche pipe.

10. The system of claim 9, wherein the shower attachment member is releasably coupled to a shower head.

11. The system of claim 9, wherein the shower attachment member wraps around the shower head.

12. The system of claim 9, wherein the shower attachment member captures all the water exiting the shower head.

13. The system of claim 9, wherein the douche pipe is configured to direct the flow of liquid toward the opening of the cavity in a rearward axial direction.

14. The system of claim 9, wherein the fluid exiting the douche pipe is under pressure such that the pressure is used to direct the expulsion of bacteria out the opening of the cavity without movement of the douche pipe.

15. The system of claim 9, wherein the douche pipe is ribbed so as to grip the cavity of an anus.

16. The system of claim 9, wherein the douche pipe is configured to allow for the ergonomical control and use by a user.

17. A douche pipe for cleansing a cavity in a living body, comprising:
a base portion having a fitting for attachment to a tube, water being permitted to flow through the fitting; and
a shaft configured to internally receive fluid passing through the fitting and configured to eject the fluid through a plurality of ports, the plurality of ports aligned so as to direct the flow of fluid rearward toward the base portion,
wherein the fluid is configured to pull bacteria and potential infections out of the cavity without requiring manipulation of the shaft within the cavity.
